(19) United States
(12) Patent Application Publication
(10) Pub. No.: US 2024/0188958 A1
(43) Pub. Date: Jun. 13, 2024

(54) NERVE CONDUIT

(71) Applicant: Tissium S.A., Paris (FR)

(72) Inventors: Marie Malafosse, Paris (FR); Simon Menand, Paris (FR); Estelle Collin, Paris (FR); Maria Pereira, Paris (FR)

(21) Appl. No.: 18/555,318

(22) PCT Filed: Apr. 26, 2022
(86) PCT No.: PCT/EP2022/061084

§ 371 Date: Oct. 13, 2023

(30) Foreign Application Priority Data
Apr. 26, 2021 (EP) 21170494.5

(51) Int. Cl. A61B 17/11; A61B 17/00

(52) U.S. Cl. CPC A61B 17/1128; A61B 2017/00004; A61B 2017/1132

(57) ABSTRACT

Nerve conduits support the repair of nerve lesions by facilitating proper insertion and/or fixation of nerve ends into the nerve conduit. A nerve conduit includes an elongate body with a central portion defining an inner cavity and end portions defining a respective opening to the inner cavity and arranged adjacent to the central portion and at longitudinally opposing ends of the elongate body. A cross-sectional area of at least one opening is larger than the cross-sectional area of the inner cavity of the central portion. A use and a method of treating a nerve lesion using such nerve conduits is also disclosed.

### CROSS-REFERENCE TO RELATED APPLICATIONS

This application is the U.S. National Stage of PCT/EP2022/061084 filed on Apr. 26, 2022, which claims priority to European Patent Application 21170494.5 filed on Apr. 26, 2021, the entire content of both are incorporated herein by reference in their entirety.

### FIELD OF THE INVENTION

The invention relates to nerve conduits as well as a use and a method of treating a nerve lesion using such nerve conduits. In particular, the invention relates to nerve conduits supporting the repair of nerve lesions by facilitating proper insertion and/or fixation of nerve ends into the nerve conduit.

### BACKGROUND OF THE INVENTION

Upon injury of a person, tissue damage may involve one or more nerve lesions of the peripheral nerve system resulting in a partial sensory loss and/or impaired motor skills. Such injuries involving nerve damage particularly occur in the upper extremities, such as a hand or finger of a person, such that a person may experience a loss e.g. in tactile or haptic feedback and/or may have difficulties in controlling fine motor skills in the injured region, if the nerve lesion is not treated properly.

Current treatments of nerve lesions include coaptation of the nerve ends by various suturing techniques so as to provide a connection between the respective nerve ends in an essentially tensionless manner. In case a more severe defect is present, wherein the nerve ends are not directly adjacent to each other, a reconstruction may be required to overcome a corresponding gap. A reconstruction may be provided e.g. by an autologous or allogenic nerve graft. Alternatively, a reconstruction may be performed by providing a tubular structure so as to provide a nerve guide. Such tubular structure may be provided e.g. by autologous or allogenic venous structures or by artificially manufactured nerve conduits made of a biocompatible material. The use of a tubular structure may furthermore facilitate the repair of the lesion irrespective of the presence of a gap, e.g., by providing further mechanical support and structural stability, providing a tensionless repair, providing guidance to axonal growth limiting the risk of neuroma, reducing an inflammatory response to the site of the lesion, and/or limiting the extent of fibrous tissue development.

The tubular structures and, in particular, nerve conduits are generally formed as an essentially cylindrical shape extending in a longitudinal direction and defining an inner lumen or through hole from one end to the opposing end. The shape may also be formed as an irregular cylindrical shape, e.g. having a cross-section resembling a star-shape or snowflake-shape, be formed as an asymmetrical cylindrical shape, or may be formed as a shape having an essentially (rounded) rectangular cross-section. The dimensioning and shape are chosen so as to be compatible with the affected microstructure of the injured nerve tissue and surrounding tissue. Accordingly, in order to treat a nerve lesion, a nerve end is inserted into the nerve conduit via one end of the nerve conduit and the corresponding other nerve end is inserted into the nerve conduit via the opposing end of the nerve conduit. Both ends may then be fixed or coupled to the nerve conduit by means of suturing techniques or by applying or depositing a medical adhesive, for example. In the implanted state, the nerve conduit hence forms a nerve guide, wherein the continuous cylindrical shape of the nerve conduit and the inner lumen provides a directional path during neurogenesis.

US 2010/0016874 describes a double-walled toroidal sheath structure used as nerve conduit having first and second apertures defined by a flexible connection between respective first and second inner and outer surfaces that are capable of eversion and un-eversion from one to the other. When a nerve end is placed at one aperture of the structure, an outside force is exerted on the conduit and the eversion and un-eversion of the inner and outer surfaces allows longitudinal roll shifts of the sheath over the nerve. When nerve ends are placed at the first and second aperture, the successive sheath shifts result in connecting them.

CN 110236622 relates to a nerve conduit for performing a small-gap (i.e. 2 mm) sleeve joint and suturing nerves of different sizes.

U.S. Pat. No. 3,833,002 describes nerve conduits comprising a tube with an inside diameter slightly greater than the diameter of the nerve ends to permit their slidable insertion. Advantageously, the conduit is adapted to enable vacuum to be applied to the interior of the tube to draw the nerve ends in direct contact or close proximity with each other before application of sealing material at the tube/nerve junction.

WO 2012/133019 describes a sleeve body for nerve regeneration that does not need microsuture or sealing material to maintain nerve ends in place. The sleeve body comprises separated arm pieces divided into a plurality of sections via slits extending from the end section of the body and one clamping body. In use, the arm pieces are displaced from a position separated from the outer circumference of the nerve to a position in contact with the outer circumference of the nerve by moving the clamping body and thus applying radial force on the arms pieces. The ends of the nerves are wrapped and securely held without the need for suturing work.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded. In the context of the present invention, the term “comprise” encompasses the term “consist of”. The term “comprising” thus encompasses “including” as well as “consisting” e.g., a composition “comprising” X may consist exclusively of X or may include something additional e.g., X+Y.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention.

The term “about” in relation to a numerical value x means x±10%.

In numerical values with decimal places, comma (“,”) or point (“.”) are used interchangeably herein, i.e. throughout the present specification and claims. Accordingly, numerical values with decimal places may be expressed with either comma (“,”) or point (“.”). For example, the exemplary value of “0.5” may also be expressed as “0.5”, which likewise applies to other values with decimal places. In particular, a comma (“,”) in a numerical value indicates decimal places, but is not used as “thousands separator”.

### SUMMARY OF THE INVENTION

Starting from the known prior art there is a need to further facilitate the repair of nerve lesions.

According to the invention it has been recognized that the dimensional limitations and the continuous cylindrical shape of current nerve conduits render it difficult to insert nerve ends into the respective ends of the nerve conduit. The nerve ends are either pushed into the nerve conduit, risking potential nerve damage, or are pulled into the nerve conduit, for example, using microsutures or vacuum, which may be cumbersome and may furthermore also be detrimental for the respective nerve ends. Furthermore, it has been found difficult to ensure that a predefined amount of a medical adhesive for the fixation of the nerve ends to the nerve guide is properly applied or deposited so as to enclose the respective nerve ends into the guide in an adequate manner. That is, a medical adhesive may not be homogeneously applied around the respective nerve end and/or an excess amount of medical adhesive may leak into a space between the respective nerve ends, potentially impairing nerve growth, or out of the conduit towards the surrounding tissue, potentially resulting in a loss of flexibility of the nerve conduit and/or negatively affecting said surrounding tissue.

It is hence an object of the present invention to further facilitate the repair of nerve lesions and, in particular, to ensure proper insertion and fixation of the respective nerve ends in and to the nerve conduit without the need of microsutures or vacuum and/or minimizing the damage to the underlying nerve.

Said object is achieved as described herein. Preferred embodiments are depicted herein, the description, and the Figures.

Accordingly, in a first aspect, a nerve conduit is suggested, which comprises an elongate body. The elongate body comprises a central portion defining an inner cavity and end portions defining respective openings to the inner cavity and arranged adjacent to the central portion and at longitudinally opposing ends of the elongate body. According to the invention, a cross-sectional area of at least one opening is larger than the cross-sectional area of the inner cavity of the central portion.

The provision of the larger cross-sectional area at least at one end of the nerve conduit significantly facilitates the insertion of a respective nerve end of a nerve to be repaired. In particular, the larger cross-sectional area may provide a tolerance of an (unintended) offset of the insertion height of the nerve end with regard to the cross-sectional area of the inner cavity of the central portion during a repair procedure. Thereby, even if such offset is provided, it is ensured that the nerve end is received within the nerve conduit and (minor) adjustments may be performed without requiring a re-insertion of the nerve end or specific action on the nerve conduit. In addition, the larger cross-sectional area may serve as an optical guiding surface for a surgeon, thus further facilitating proper insertion of the respective nerve end. Thereby, proper insertion and placement of the nerve end into the nerve conduit, e.g. into the inner cavity, which may be particularly dimensioned to accommodate the nerve ends to be connected, may be achieved more easily and in a time-effective manner and without adversely affecting the respective nerve end.

At the same time the nerve conduit may still be configured according to the desirable or predefined dimensions and may provide a continuous guiding structure for neurogenesis. Modifications of the central portion are hence not necessary, such that a particular structural stability and dimensioning of the nerve conduit may be achieved. The lumen or interior of the elongate body, i.e. an inner wall of the central portion and/or end portion(s), may, however, comprise grooves, internal structures and/or holes. This may avoid a pressure build-up or a potential air bubble formation inside the nerve conduit and/or may improve the mechanical properties of the nerve conduit and/or promote nerve growth.

By the same token, depending on the material used for the nerve conduit, a predefined flexibility and/or resilience may be provided for at least the central portion, such that the end portions do not (significantly) affect the overall structural characteristics of the elongate body or the nerve conduit as a whole.

The larger cross-sectional area furthermore can assist in applying a medical adhesive towards or outside of the inner cavity, which may be particularly dimensioned to accommodate the nerve ends to be connected, via the corresponding opening, preferably only into a respective opening. For example, the larger cross-sectional area may prevent that the medical adhesive is inadvertently applied to an exterior surface of the nerve conduit and/or within the inner cavity of the central portion. In addition, this may provide that the medical adhesive is directed towards an interface between the opening and the inner cavity and/or that, when applying or inserting a predefined amount of medical adhesive, no significant amount of the medical adhesive leaks out of the nerve conduit and/or into the inner cavity of the central portion. In particular, the larger cross-sectional area facilitates applying a medical adhesive at the junction between the nerve and the nerve conduit, i.e. at the junction between the inserted nerve end and an edge of the opening at an end portion of the opening facing away from the central portion. The larger cross-sectional area may also be provided to receive a particular amount of the medical adhesive e.g. during inadequate application, such that it may indicate that the predefined amount of medical adhesive has been applied or it becomes immediately apparent that an application error occurs during the application of the medical adhesive. In other words, the larger cross-sectional area may also be configured to indicate that an excess amount of medical adhesive is being applied or the medical adhesive is not properly applied, such that the application needs to be interrupted or stopped.

According to a preferred embodiment:
the contact surface between the medical adhesive and the end portions extending from the central portion and having larger cross-sectional area, and/or
the anchoring of the medical adhesive over the end portions extending from the central portion and having the larger cross-sectional area,
is increased by means of external structures with particular geometries or surface irregularities (such as holes, grooves or threads) on the outer surface of the end portions extending from the central portion.

The nerve conduit may have a variety of dimensions and shapes and may be configured to repair one or more nerve lesions. For example, directly adjacent nerves may be repaired by a single nerve conduit, wherein the inner cavity and the openings may e.g. provide a separating wall, such that two adjacent nerves may be respectively received in a separated compartment.

Accordingly, the elongate body may comprise multiple end portions and/or the openings at the longitudinal ends of the elongate body may comprise multiple openings at least one of its end portions, e.g. two distinct openings, or may be configured as a single opening defining two separate compartments (or receiving portions) for enabling insertion of and accommodating a respective nerve end. Such embodiment may have the advantage that only a single nerve conduit is required for multiple nerve lesions and/or nerve extensions and the procedure for repairing the nerve lesions or connecting the respective nerve ends may be facilitated. The elongate body may e.g. have a Y-shape.

In some embodiments, the nerve conduit comprises a single inner lumen, in particular a single inner cavity only. A single inner cavity is sufficient for a variety of application, provides flexibility and enables a simple production and handling. Preferably, the central portion is thus formed as an essentially tubular shape. In other words, the central portion of the nerve conduit is preferably a tube, such as a cylindrical tube, with a single inner cavity, which may have essentially the same thickness (essentially the same inner diameter and essentially the same outer diameter) along the entire length of the central portion. Preferably, the elongate body (of the nerve conduit) comprises two end portions, in particular one end portion at either end of the central portion (tube). The shape, the dimensions and/or the material (and/or other features) of the two end portions (of the same nerve conduit) may be essentially the same. Accordingly, the two end portions (of the same nerve conduit) differ preferably only in that they may be arranged (at either end of the central portion) in mirror-symmetrical manner (along the longitudinal axis of the central portion). By such configuration the structural complexity of the overall nerve conduit may be reduced and the nerve conduit may be dimensioned so as to be adapted to the site of implantation and/or the requirements with respect to the particular nerve lesion. Such configuration may furthermore facilitate insertion of the respective nerve end at the opening having the larger cross-sectional area, since the elongate body may be configured only for a single nerve and inadvertent misplacement into a compartment not corresponding with the respective nerve end to be connected may be effectively avoided.

The tubular shape may provide that continuous outer dimensions may be provided, which may be advantageous for implanting the nerve conduit with regard to the surrounding tissue. Furthermore, the tubular or cylindrical shape may provide sufficient structural stability and may prevent sharp bends or kinking during tissue movement, i.e. compression or extension. The tubular shape may accordingly also provide that a homogeneous structure is provided, which reacts in a predefined manner along the entire central portion when forces act upon the central portion, e.g. upon impact.

The tubular structure may furthermore essentially correspond to the shape of the inner cavity, such that a continuous guiding structure for neurogenesis or nerve growth is provided, facilitating proper growth and connection of the respective nerve ends without providing an undesirable biasing and while achieving that the dimensioning of the inner cavity may be kept to a desirable and/or required minimum.

The openings and inner cavity may define a single lumen or continuous through hole. In other words, within the elongate body, a channel may be provided, which extends from one end of the elongate body to an opposing end of the elongate body in the longitudinal direction so as to provide a (fluid) connection or communication between the exterior of the respective ends via the inner cavity. The inner cavity should hence be understood as a central lumen, which extends and opens towards the respective end portions, such that a nerve end may be introduced into the lumen or through hole via the openings of the respective end portions. As described in the above, sections of the lumen or the elongate body may comprise holes, pores, grooves or particular geometries or surface irregularities, and/or a filler, which may facilitate the insertion, retention and/or growth of the respective nerve ends. One or more of these features may either be arranged in the interior or at the exterior of the elongate body or may extend between the interior and exterior of the elongate body. Furthermore, the elongate body and, in particular, the lumen may contain a drug, e.g. by means of coating or integrated in the material of the elongate body, which may be released over time and may e.g. facilitate nerve growth.

Preferably, the cross-sectional area of each of the openings is larger than the cross-sectional area of the inner cavity. This has the advantage that insertion and/or fixation of each of the respective nerve ends into the nerve conduit may be facilitated, thereby further supporting the repair procedure of the respective nerve lesion. The central portion may also be defined by an interface region between respective end portions and/or openings, wherein the longitudinal or axial extension of the central portion is essentially negligible compared with the corresponding extension of the end portions and/or openings, e.g. approximating zero or being less than e.g. 10 percent of the smallest axial extension of a respective opening or end portion. At least one of the openings may have a cross-sectional area being distinct from at least one other opening at an opposing end of the central portion and/or may have a cross-sectional area corresponding to the cross-sectional area of the inner cavity of the central portion, thereby forming an asymmetric nerve conduit.

Although the larger cross-sectional area of the at least one opening is already advantageous in terms of the insertion and/or fixation of the respective nerve end(s), the cross-sectional area of the at least one opening preferably increases in the longitudinal direction and away from the central portion. In other words, the opening may increase in a direction extending away from the central portion and along the longitudinal direction. In case of a tubular structure of the central portion, i.e. having a circular cross-section, a radial extension may hence be increased, preferably in all radial directions, whereas for an ellipsoid structure of the central portion, a radial extension may be increased in at least one direction, i.e. maintaining an overall ellipsoid shape of the opening or transforming to a more circular shape of the opening. Preferably, an increase of the opening of the respective end portion is provided directly starting or extending directly from the central portion.

The increasing opening may have a variety of shapes. Although a radial flare at the interface between the central portion and the respective end portion may be advantageous e.g. for handling of the nerve conduit during implantation, a gradually increasing and/or stepless increase of the opening may be preferred to facilitate insertion of the respective nerve end and/or to provide a guiding surface during the insertion of the respective nerve end. In other words, a gradual increase of the opening or cross-sectional area may direct the nerve end that is inserted into or towards the inner cavity and may avoid that a bending or sharp folding occurs by properly deflecting or guiding the respective nerve end. Hence, such gradual increase in the cross-sectional area may avoid a stump trauma or any other adverse effects of the respective nerve end potentially caused when the nerve end is brought into contact with an orthogonal and/or straight surface.

Accordingly, the at least one end portion and the corresponding opening are preferably formed as a rotationally symmetric shape along a longitudinal axis defined by the elongate body, wherein said shape is an essentially U-shape, sigmoidal shape, conical shape, concave shape, funnel shape, or parabolic shape.

As outlined above, such shapes may provide a gradual increase of the cross-sectional area. The preferred shapes are to be understood as a shape essentially extending from the central portion and as seen in a longitudinal sectional view of the elongate body. They are preferably continuous in the circumferential direction, i.e. do not form gaps, based on their rotational symmetry. The particular shapes may facilitate that the respective nerve end is advanced into the opening and further into the inner cavity in an essentially stress-free and tensionless manner, since a contact between the nerve end and the inner surface of the end portion does not result in a significant bending of the nerve, but instead deflects the nerve end or tip thereof towards the advancing direction, i.e. towards the inner cavity. The hence provided guiding surface or structure facilitates the insertion procedure for a surgeon while at the same time the inner surface of the end portion does not adversely affect the respective nerve end during insertion.

Particularly advantageous embodiments in this regard include the funnel shape and/or the parabolic shape of the end portion and corresponding opening since these shapes may provide an essentially continuous and radially inward directing guiding surface, i.e. towards the inner cavity of the elongate body. Furthermore, these shapes provide further improved structural stability while avoid steps or sharp edges or large differences in the cross-sectional area towards the inner cavity or directly at the intersection between the inner cavity and the opening extending therefrom. The funnel shape may be formed e.g. essentially conical or have a parabolic extension so as to achieve e.g. the same maximum cross-sectional area compared with a conical shape yet in a corresponding smaller longitudinal extension and/or at a reduced angle with the central portion.

At least one of the end portions may also comprise a star shape as seen in a cross-sectional view of the end portion and which is preferably provided in a rotationally symmetric manner. Accordingly, a wall of the elongate body at the end portion may have two or more rounded bulges radially extending the opening, which may be equally spaced apart to each other in the circumferential direction. The bulges hence form inner and outer grooves respectively defined by a circumferential width and radial extension of a respective bulge or a spacing between two bulges. Such shape may also facilitate the insertion of a corresponding nerve end by increasing the respective opening and may furthermore facilitate handling the respective end portion by providing an improved gripping surface during surgery.

While the star shape may hence already provide an increased opening on itself, such star shape may also be provided as a gradually increasing cross-sectional area and may hence be combined e.g. with a funnel and/or parabolic shape of the respective end portion. Furthermore, such shape is not limited to the respective end portion, but may likewise be provided for the elongate body in its entirety, such that opposing end portions and a central portion may be formed in the same manner. This further facilitates handling during implantation and surgery and may be advantageous in terms of manufacturing. These above described preferred shapes and, in particular, the funnel and/or parabolic shape, furthermore facilitate the application of a medical adhesive, e.g., by maintaining the applied medical adhesive essentially outside of the inner cavity and/or by forming a junction or outer edge or rim to apply the medical adhesive, thereby ensuring that the respective nerve end is held within the nerve conduit and preventing a leakage towards the surrounding tissue and/or into the inner cavity of the central portion. The shapes may also define a limited reservoir or recess, preventing the medical adhesive from entering the nerve conduit and ensuring that the medical adhesive is held at said junction or edge region, e.g. in case of an application of excess medical adhesive or incorrect application. Furthermore, grooves formed by a cross-sectional star-shape of a respective end portion may facilitate that a contact surface with a medical adhesive may be increased, thereby improving the securing of a respective nerve end to said end portion.

The end portions may have been adapted to a particular dimension of a nerve lesion and the nerve conduit as a whole may be dimensioned to provide a bridging structure to a corresponding distance between respective nerve ends. Accordingly, the end portions of the elongate body may be configured differently so as to provide different characteristics, e.g. to accommodate different dimensions of the corresponding nerve ends. However, it is preferred that the end portions are equally formed. In such manner, the nerve conduit may be implanted essentially independent of the orientation of the respective end portions. In other words, by providing equally shaped end portions the nerve conduit may be configured to be essentially mirror symmetric and the nerve conduit may hence be placed in either direction, i.e. may also be placed in a 180 degrees inverted orientation.

The inner cavity and the openings are preferably formed by a single wall of the elongate body defining an inner diameter and outer diameter of the elongate body. In such a way, the structural complexity of the nerve conduit may be reduced while a more robust nerve conduit may be provided that responds to forces acting upon the elongate body in a predefined and/or expectable manner. For example, compressive and/or tensile forces acting on the elongate body may be better distributed (or absorbed) by the single wall, such that focal points of stress or tension may be avoided. Furthermore, depending on the material of the elongate body, a more homogeneous deflection of the nerve conduit may be provided, e.g., when the elongate body is formed of a wall having elastic and/or resilient characteristics.

For particular situations, the wall may have a (gradually) reduced thickness in at least one of the end portions and starting from the central portion towards the (longitudinal) outermost end of the respective end portion. In such a way, the larger cross-sectional area of the opening may be defined by the wall thickness.

However, the wall preferably comprises an essentially continuous thickness along the circumferential and longitudinal direction of the elongate body. This not only facilitates manufacturing of the nerve conduit, but also ensures that similar structural characteristics are provided along the longitudinal and circumferential direction. Contrary to a beveled or chamfered end portion, the provision of a continuous wall thickness also facilitates end portions with gradually increasing diameters and/or cross-sectional areas over a larger longitudinal extension due to the improved structural characteristics. Such continuous thickness may hence also be provided by a minimum continuous thickness throughout the entire longitudinal extension of the elongate body, while the thickness may exceed the minimum extension at particular portions, preferably at one or more end portions. Furthermore, adequate placement of the nerve conduit is hence also not dependent on an orientation of the nerve conduit in a circumferential direction.

In order to provide an essentially homogeneous inner cavity, which is advantageous to provide optimal nerve growth conditions and to provide an adequate guiding of the growing nerve end without undesirable biasing, the inner diameter and outer diameter of the central portion are preferably essentially continuous in the longitudinal direction of the elongate body.

The diameter of the at least one opening may increase in the longitudinal direction away from the central portion. In other words, the larger cross-sectional area of the corresponding opening may be defined by the inner diameter of the wall, wherein the diameter is enlarged towards the longitudinal end of the end portion facing away from the central portion and/or (directly) starting from the central portion. The increase in the opening is preferably gradual and homogeneous in all radial extensions. For example, the opening may have a circular cross-section along the entire longitudinal direction of the respective end portion, wherein the diameter of the circular shape is gradually increased. Such shape furthermore has the advantage that during implantation the placement of the nerve conduit is independent from the rotational orientation. Other shapes, such as ellipsoids, may, however, also be provided, wherein the (gradual) increase in the opening is provided by an increase in at least one radial extension.

The largest or maximum diameter of the at least one opening may be chosen such that the insertion of the respective nerve end is sufficiently supported while the overall thickness of the nerve conduit is maintained within physiologically acceptable limits. Hence, the maximum diameter is chosen to avoid that pressure and/or friction points are established with regard to the surrounding tissue and that the accommodated nerve end is sufficiently supported by the end portion.

Accordingly, a ratio between the maximum diameter of the at least one opening and the diameter of the inner cavity is preferably between 1.05:1.0 and 1.5:1.0, preferably between 1.05:1.0 and 1.2:1.0. It has been found that the difference in diameter and hence in cross-sectional area may thus be relatively small. The preferred ratio nevertheless achieves that a further improvement is provided with respect to the insertion and/or fixation of the respective nerve end. With a smaller ratio the application of a medical adhesive is furthermore facilitated, since this further reduces the risk of applying the adhesive between the respective nerve ends in the inner cavity of the central portion.

Preferably, the maximum (inner) diameter of the at least one opening is smaller than or corresponds to the outer diameter of the central portion. That is, seen from a longitudinal section, the opening does not exceed the outer diameter of the inner cavity in a radial direction. Thereby, depending e.g. on the thickness of the wall and the dimensioning of the inner cavity, the maximum diameter of the opening may hence be relatively small so as to maintain the overall appearance and/or to avoid large radial protrusions from the central portion.

The outer surface of the end portions extending from the central portion is preferably aligned with the outer surface of the central portion. In some embodiments, the outer surface of the end portions extending from the central portion may be free of steps or edges with the outer surface of the central portion. According to a preferred embodiment, the outer surface of the end portions extending from the central portion may also support or comprise external structures with particular geometries or surface irregularities shaped to form retention surfaces, such as holes, grooves or threads.

The maximum outer diameter of the end portion(s) is preferably larger than the (maximum) outer diameter of the central portion. Preferably, the outer end of the end portion (which constitutes the end of the nerve conduit) is provided by a continuous edge, preferably having (in a cross-sectional view) an essentially circular or ellipsoid shape. In particular, the edge forming the outer end of the end portion does preferably not contain any recesses or cut-outs. Thereby the nerve can be inserted into the nerve conduit more easily. In some embodiments, (the outer surface of) the end portion may have essentially the shape of a truncated cone.

In particular, the minimum outer diameter of the (truncated-cone-shaped) end portion(s) may correspond to the outer diameter of the (tube-like, e.g. essentially cylindrical) central portion. Thereby, a smooth transition between central portion and end portion may be provided on the outside of the nerve conduit. The minimum inner diameter of the (truncated-cone-shaped) end portion(s) may correspond to the inner diameter of the (tube-like, e.g. essentially cylindrical) central portion. Thereby, a smooth transition between central portion and end portion is provided on the inside. In this way, the nerve conduit may have enlarged ends (as compared to the central portion) for easy insertion of the nerves, while the thickness of the wall of the nerve conduit may be kept to a minimum. This provides good flexibility and less material may be used. In some embodiments, the nerve conduit may have essentially the same thickness of the wall over its entire length, i.e. in the end portion(s) as well as in the central portion.

The maximum (inner and outer) diameter of the (truncated-cone-shaped) end portion(s) typically constitute the end(s) of the nerve conduit. Preferably, there is no (second) cylindrical portion adjacent to the larger end of the (preferably truncated-cone-shaped) end portion(s).

In a preferred embodiment, the inner cavity of the central portion defines a single lumen (preferably the nerve conduit comprises a central portion having an essentially tubular shape (with a single inner cavity)) and the nerve conduit comprises two end portions, which are located at either end of the central portion, wherein the maximum outer diameter of the end portions is preferably larger than the outer diameter of the central portion and the outer end of the end portion (which constitutes the end of the nerve conduit) is provided by a continuous edge (without any recesses or cut-outs). Preferably, the two end portions differ from each other only in that they are arranged (at either end of the central portion) in mirror-symmetrical manner (along the longitudinal axis of the central portion).

To accommodate for a variety of lengths to be bridged by the nerve conduit, the nerve conduit may be formed with different dimensions. The length of the central portion and the length of an end portion (or of each end portion) in the longitudinal direction may hence be different from each other and for each predefined configuration. Preferably, a ratio between the length of the central portion and the length of an end portion in the longitudinal direction is from 1.2:1.0 to 15:1.0; preferably from 1.2:1.0 to 12:1.0; more preferably from 1.2:1.0 to 10:1.0. In some embodiments, the ratio between the length of the central portion and the length of an end portion in the longitudinal direction is preferably between 1.2:1.0 and 6.0:1.0, more preferably between 1.2:1.0 and 1.4:1.0 or between 1.8:1.0 and 2.2:1.0 or between 4.4:1.0 and 5.4:1.0.

The ratio may be dependent on the overall length or absolute length of the nerve conduit or elongate body. Larger ratios may e.g. be provided for conduits having relatively small dimensions. In particular, the above larger ratios, i.e. of between 4.4:1.0 and 5.4:1.0 may be provided for nerve conduits having smaller dimensions, wherein hence relatively small end portions are provided compared with the central portion.

By the same token, a ratio between the length of the central portion and the length of the elongate body in the longitudinal direction may be from 0.3:1.0 to 1:1.0; for example between 0.3:1.0 and 0.8:1.0. Such ratio may increase with a decreasing overall length of the elongate body, such that the length of the elongate body may be primarily defined by the central portion and only includes small end portions for nerve conduits having relatively small or smallest dimensions.

The dependence of the length of the end portions, and the central portion, on the overall length may also provide that longer end portions may be provided when bridging longer distances between corresponding nerve ends, e.g. due to trauma, wherein the longer end portions may extend along a corresponding longer part of the respective nerve end. This may provide further structural stability, since these end portions may be configured to accommodate a (larger amount of) medical adhesive.

The length of the central portion in the longitudinal direction may be from 3 mm to 40 mm, preferably from 4 mm to 30 mm, more preferably from 4.5 mm to 25 mm, for example between 5 mm and 10 mm or between 6.5 mm and 8.5 mm. The length of the elongate body in the longitudinal direction may be from 5 mm to 50 mm, preferably from 6 mm to 40 mm, more preferably from 7 mm to 30 mm, for example between 7 mm and 25 mm or between 9 mm and 22 mm. The length of (each of) the end portion(s) in the longitudinal direction may be between 1 mm and 8 mm, preferably between 1.3 mm and 6.5 mm. These dimensions have been found to be particularly advantageous to bridge nerve defects or lesions while facilitating nerve end insertion and/or fixation.

The diameter of the inner cavity may be from 1 mm to 15 mm, preferably from 1 mm to 12 mm, for example between 1 mm and 12 mm or between 1.5 mm and 6.5 mm. The maximum diameter of the at least one opening may be from 1 mm to 15 mm, preferably from 1.5 mm to 13 mm, for example between 1.5 mm and 7.5 mm, more preferably from 1.75 mm to 7 mm, for example between 1.75 mm and 6.5 mm. Again, such dimensions have been found to be particularly advantageous to provide the required support for the nerve ends and nerve growth while facilitating nerve end insertion and/or fixation.

The elongate body may be formed of a biocompatible material, an inert material, a bioimplantable material, and/or a biodegradable material. The material may be chosen so as to provide a predefined structural stability while essentially avoiding or at least reducing the inflammatory response of a patient to be treated. For example, a biocompatible material may be chosen, which is gradually degrading over time after implantation yet which may initially provide sufficient structural support to adequately repair a nerve lesion and ensure that the respective nerve ends are connected properly and with sufficient stability, e.g. during movement of the tissue.

The particular material may furthermore be chosen in order to facilitate or support nerve growth, for example, by comprising or otherwise incorporating or including a corresponding coating, e.g. with a biologically active agent and/or one or more neurotrophic factors. Other examples of such biologically active surface functionalities include, but are not limited to, e.g. anti-inflammatories, immunosuppressants, and neuroprotective agents. Biologically active agents may be surface bound and/or be entrapped in a structure defining the elongate body, e.g. the wall described in the above. Examples of such biologically active agents are cytokines, nerve growth factor, hyaluronic acid, tacrolimus, cyclosporin A, melatonin, vitamin B12, methylprednisolone, riluzole, taxol, cetuximab; preferred example is tacrolimus.

Preferably, the elongate body is formed of a polymer-based material, preferably an elastomer. This has the advantage that a plurality of manufacturing methods may be applied and/or particular characteristics often material may be provided based on e.g. a polymer unit. In particular, the polymer-based material may be a biocompatible material, which furthermore has elastic properties, such that, in the implanted states, the nerve conduit may adapt to tissue movement surrounding the repaired nerve lesion.

The elongate body may be formed of a polymerized and/or crosslinked polymer unit comprising an ester group component and an acid ester group component, the ester group component preferably being a polyol and the acid ester group component preferably being a polyacid.

The material being used for the central portion may be the same as the material of the end portions. Thereby, manufacturing may be further facilitated and structural characteristics of the elongate body may be essentially homogeneous along the longitudinal direction of the nerve conduit. In this matter, if the nerve conduit is configured accordingly, biodegradation (and/or bioresorption) may also occur in a predefined or expected manner. However, it may also be provided that at least one of the end portions is formed of a material being different from the central portion and/or the opposing end portion. Such configuration may be advantageous e.g. if one end portion has a particular functionality and may require different characteristics, e.g., if such an end portion is configured as a primary inlet for the application of a medical adhesive.

Preferably, the central portion and the end portions are integrally formed or formed of a single piece. In other words, the elongate body preferably does not require particular connections between the central portion and the end portions. Instead, it provides a material bonding, provided by the material off the central portion and/or the end portion, and corresponding structural integration of the respective elements. By providing the elongate body as a single piece, the robustness of the nerve conduit may be further improved since separate connections between components are effectively avoided.

The nerve conduit may be formed by a 3D-printing process. This is particularly advantageous when the material of the nerve conduit is polymer-based (as described, for example, in WO2019/180208), wherein a curing of the material may be provided essentially instantaneously, for example using (UV) light. Furthermore, this provides an accuracy level that may not be (easily) achieved by means of extrusion and/or a dipping process. In particular, the 3-D printing process enables to obtain a particular shape of the nerve conduit, wherein, for example, the printing process may provide that particular biologically active agents are integrated in the 3-D structure according to a predefined pattern, e.g. within a mesh structure and/or in particular pockets or cavities formed by the 3-D structure. Thereby, orchestration and support of the nerve repair may be further improved and/or biodegradation may be achieved in a more controllable matter.

In some embodiments, the nerve conduit of the present invention may be combined in use with a device for applying adhesive (applicator), such as the device for applying adhesive as described in WO 2022/048799 A1.

In order to ensure that a relative movement between a medical adhesive and a respective end region is reduced or even avoided under normal physiological behavior, a securing of the medical adhesive to the nerve conduit may be advantageous. Accordingly, at least one of the end portions may comprise one or more retention surface(s) at an outer surface of the elongate body, in particular of the end portion(s), wherein the retention surface(s) is/are configured in particular for securing a medical adhesive to the respective end portion. Thereby, the medical adhesive that has been applied to the respective end region and/or a corresponding nerve end may remain in situ without any significant movement, even after curing of the medical adhesive. In other words, by means of the retention surfaces, the medical adhesive may be held in place, preferably in a form fitting manner with the one or more retention surfaces. The retention surfaces may provide that a contact surface with the medical adhesive is increased. Furthermore, it may provide that a predefined surface roughness is provided, thereby improving the efficacy of the medical adhesive to bond with the respective end region. Accordingly, a loosening or slipping of the medical adhesive may be effectively prevented by means of the shape and/or surface of the retention surface. In some embodiments, the (outer surface of the) end portion(s) is structured, i.e. it comprises a structure. In particular, such a structure of the (outer surface of the) end portion(s) forms the “retention surface”. Advantages of the end portion(s) having a structure or retention surface include (i) to increase the contact between the surface of the end portion and the adhesive/guide; and (ii) to create anchor points for the adhesive.

In some embodiments, he retention surface(s) of a respective end portion may be formed as a plurality of (e.g., ellipsoid or circular) holes, that may be arranged in at least one row in a circumferential direction of the elongate body. The holes may hence be positioned in a linear fashion along the circumference of the elongate body. The number of rows preferably ranges between 1 and 10 rows. Each row preferably comprises 2 to 20 holes, wherein the holes may advantageously be equally spaced apart in the circumferential direction of the elongate body. Preferably, the holes are arranged in 2 to 4 rows and/or each row comprises 4 to 8 holes, wherein the holes of adjacent rows are preferably arranged in a staggered formation. For example, two rows may be provided at a respective end region, wherein each row may comprise e.g. six holes.

The number of holes and rows as well as their arrangement have been found to be advantageous in order to provide and maintain the required structural stability, while simultaneously providing an improved hold for securing a medical adhesive to the nerve conduit. Furthermore, such embodiments may exhibit a rotational symmetry, which may facilitate the correct insertion and placement of the nerve conduit at the target tissue site. In certain embodiments, the size or diameter of the holes may be between 50 μm and 750 μm and, for further improved structural stability, preferably ranges between 150 μm and 600 μm and may particularly be about 500 μm.

Each hole may be formed as a cut-out, a recess, or a through-hole in a wall forming the elongate body. Thereby, an improved securing of the medical adhesive may be provided. In particular, a through-hole may be further advantageous. Such a preferred through-hole may facilitate that the medical adhesive surrounds the elongate body at the respective end portion, in particular both, at the exterior and in the interior of the nerve conduit, which may depend on the chosen dimensions and/or, in particular, on the diameter of the holes. By the same token, this may also provide that the medical adhesive is brought into contact with the respective nerve end to be inserted. The holes may hence have a radial height or depth corresponding to a strength of a wall defining the elongate body, but may also have a reduced height. Preferably, the holes have a height or depth of between 40 μm and 60 μm or about 50 μm.

In some embodiments, the one or more retention surface(s) of a respective end portion may be preferably formed as at least one groove extending in a helical direction along a longitudinal axis defined by the elongate body. The helical shape may define a thread extending along the outer surface of the respective end portion.

Although the one or more grooves may define sharp edges, the at least one groove preferably comprises rounded edges. The rounded edges are to be understood such that at the interface with the top outer surface of the respective end portion and at an opposing bottom surface of the groove with a wall defining the elongate body no straight (angled) edges or steps are present, but instead a gradual transition is provided at corner surfaces of the groove. By means of the rounded edges, stresses within the material of the elongate body may be reduced, resulting in a reduced occurrence of rupture or breakage. Accordingly, the provision of rounded edges may also reduce the required thickness of a wall defining the elongate body at least at the end portion to support the groove.

Alternatively, or (preferably) additionally to the round edges, the at least one groove may define at least one undercut. The provision of an undercut may improve the anchoring of the medical adhesive to the respective end portion. The undercut may be formed such that a bottom portion of the groove is at least partially covered by the outer surface of the wall. That is, the outer wall of the elongate body may at least partially extend over the groove in a longitudinal direction. Preferably, such extension forms an angle (between the bottom and the sidewall of the groove) of between 45 degrees and 90 degrees, preferably between 60 and 85 degrees, more preferably between 70 degrees and 80 degrees, even more preferably about 75 degrees. Thereby, a form-fitting or positive locking between an applied medical adhesive and the respective end portion may be improved.

In order to reduce the radial extension of the elongate body and the nerve conduit as a whole, the at least one groove may define an outermost edge of the respective end portion in a longitudinal direction of the elongate body. The groove may hence end at the end surfaces of the end portion opposing the side of the central portion and extend along the entire circumference at said end surface such that the radial extension may be reduced at said end. Thereby, the overall dimensioning of the nerve conduit may be reduced. Alternatively, the groove may also end at a longitudinal offset to said end surface, which may be advantageous for further improving structural stability.

The depth or thickness of the groove may be essentially constant along the circumference and throughout its longitudinal extension. In an alternative embodiment, the at least one groove may comprise a radial depth varying in accordance with an increase of the cross-sectional area of the at least one opening in the longitudinal direction and away from the central portion. According to a preferred example, the inner radius of the groove may be maintained continuous whereas the outer radius of the groove may increase corresponding to an increase in the total size of the opening, when a thickness of the corresponding wall portion increases in the same manner.

The extension and/or the angle of the at least one groove may vary. Preferably, the at least one groove extends between 0.5 and 10 revolutions around a longitudinal axis defined by the elongate body, thereby providing a helical structure (at the end portion(s)). Preferably, the groove extends for more than one revolution around the longitudinal axis defined by the elongate body. In particular, the number of revolutions may be between 2 to 6, preferably 3 to 5 or 4.

In some embodiments, the end portion (or each of two end portions) may comprise a single groove. In other embodiments, the respective end portion(s) may also comprise at least two grooves. In particular if two or more grooves are provided in the same end portion, each groove may extend between 0.5 and 5 revolutions around a longitudinal axis. The at least two grooves may be extending in parallel, for example forming two or more parallel (non-intersecting) helices. Thereby, arc length, curvature and torsion of the parallel grooves/helices are preferably the same, such that they only differ in their location (on the end portion). Preferably, the distances between two or more parallel grooves/helices are regular (i.e. about the same). In some embodiments, at least two grooves may extend in opposing circumferential directions and intersect each other. Both embodiments may also be combined, i.e. with two or more grooves extending in parallel and two or more additional grooves extending in opposing circumferential directions, optionally also in parallel (such that the parallel intersecting grooves provide a “checkered” or “pineapple” pattern). The number of grooves in each circumferential direction may be between 1 and 10 and preferably is between 6 and 8 or 7, depending on the longitudinal extension of the respective end portion and the angle of the grooves. The number of grooves is furthermore preferably equal for each circumferential direction. Moreover, at a single end portion (or at each end portion), the grooves/helices preferably differ only in their direction (and location on the end portion), but preferably not in other helix parameters. In other words, arc length, curvature and torsion may be essentially the same for all grooves/helices at a certain end portion (except for the direction of intersecting helices). By providing a plurality of grooves (e.g., two or more grooves) in each circumferential direction a diamond-shaped or rhombic-shaped pattern is provided at the outer surface of the respective end portion, which may resemble a pineapple surface. Thereby, a plurality of retention surfaces with a large number of edges may be provided, which may be advantageous for securing the medical adhesive to the respective end portion.

In some embodiments, the one or more retention surface(s) of a respective end portion may be formed as one or more circumferential ribs (elongated protrusions) extending from an outer surface of the elongate body. The extension of the one or more ribs may be linear to the circumferential direction or comprise an offset to the circumferential direction in a longitudinal direction of the elongate body. Preferably, in case of a plurality of ribs, e.g. between 3 and 6, at the respective end portion, the ribs may be equally spaced apart from each other and do not intersect, i.e. are preferably arranged in parallel to each other.

The one or more retention surfaces may generally be provided on a thickened wall portion at the respective end portion that is adapted to the implementation of the desired retention surface and the structural requirements at the end portion. Preferably, the thickness of the wall portion at the respective end portion may be between 1.0 and 3.5 fold the thickness of the adjacent central portion. For example, if the wall thickness of the central portion is about 200 μm, the thickness of the wall at the end portion may be between 200 μm and 700 μm and preferably is between 300 μm and 500 μm or 400 μm, i.e. comprises an added thickness of between 100 μm and 300 μm. In another example, if the wall thickness of the central portion is about 20 μm, the thickness of the wall at the end portion may be between 20 μm and 70 μm and preferably is between 30 μm and 50 μm or 40 μm, i.e. comprises an added thickness of between 10 μm and 30 μm. The thickened wall portion may be brought flush with the outer surface of the central portion by appropriate rounding or provision of fillets.

By the same token, the one or more holes and, in particular, grooves may also be provided as positive features at the outer surface of the wall of the elongate body having an otherwise essentially continuous thickness. The wall may hence comprise positive protrusions or build-ups at the respective end portion defining the respective groove or hole while the central portion does not comprise such positive features. In such case, the outer surface of the wall hence preferably defines the inner radius of the respective groove or hole.

In order to increase the efficacy of a medical adhesive and, preferably, a form-fitting or interlocking of the medical adhesive with the nerve conduit, the retention surfaces may furthermore be at least partially comprised on a portion of the central portion directly adjacent to the respective end portion. For example, at least one row of holes may be provided on the central portion, e.g. in a staggered formation to the adjacent row on the end portion, or one or more grooves may extend over the directly adjacent portion of the central portion, e.g. originate from or terminate into the central portion. Such extension of the retention surfaces may provide a gradual transition of the retention surfaces while improving the load distribution on the elongate body.

It may be provided that only one respective end portion of the nerve conduit is provided with the one or more retention surfaces. Preferably, all end portions (of the same nerve conduit) and particularly, all of the opposing end portions, e.g. two opposing end portions, are provided with one or more retention surfaces. The end portions (of the same nerve conduit) may have different types of retention surfaces, i.e. one end portion may comprise a groove while another end portion comprises a plurality of holes. Preferably, however, the end portions (in particular of the opposing ends or of all ends of the nerve conduit) have equally formed retention surfaces. The retention surfaces of opposing end portions may be mirrored about a transverse mid plane of the elongate body, for example such that they are aligned in opposing directions. For example, each of the opposing end portions (of the same nerve conduit) may comprise a single or more grooves/helices as described above, wherein the grooves or helices of the different end portions may essentially correspond to each other (e.g., having the same number and/or pattern), but wherein the different end portions (of the same nerve conduit) differ from each other in that the grooves/helices are formed in a mirrored arrangement about a transverse mid plane of the elongate body, such that they are aligned in opposing directions. For example, on one end portion, the groove may extend in clockwise direction, while on the other end portion (of the same nerve conduit) it extends in counter-clockwise direction. Alternatively, however, the retention surfaces of the different end portions of the same nerve conduit may also be oriented or arranged in the same direction, e.g. have a groove or thread in the same (clockwise or counter-clockwise) direction.

The above object is furthermore achieved by use of a nerve conduit described in the above for repairing, supporting, and/or guiding neural tissue, in particular for repairing a peripheral nerve lesion. Furthermore, the nerve conduit may be used in combination with a medical adhesive.

According to another aspect of the invention, a method for treating a peripheral nerve lesion is suggested, comprising the steps of:
providing a nerve conduit as described in the above;
inserting one end of the lesioned nerve into the nerve conduit via an opening having a larger cross-sectional area than the inner cavity of the central portion;
inserting another end of the lesioned nerve via an opening at an end portion at a longitudinally opposing end of the elongate body; and
securing the lesioned nerve ends within the elongate body.

Preferably, the securing of the lesioned nerve ends is performed by applying a medical adhesive outside of the inner cavity and/or within the openings via at least one of the openings.

Medical adhesive according to the invention may be any medical adhesive of the art. In some embodiments, the medical adhesive is able to polymerize when exposed to light. Before such polymerization the medical adhesive may be fluid or viscous.

In some embodiments of the invention, the medical adhesive comprises a pre-polymer comprising a polymeric unit of the general formula (-A-B-)n, wherein A represents a substituted or un-substituted ester, B represents a substituted or unsubstituted acid ester comprising at least two acid ester functionalities, and n represents an integer greater than 1.

Component A may be derived from a polyol, such as a diol, triol, tetraol or greater. Suitable polyols include diols, such as alkane diols; triols, such as glycerol, trimethylolpropane, triethanolamine; tetrads, such as erythritol, pentaerythritol; and higher polyols, such as sorbitol. Unsaturated diols, such as tetradeca-2,12-diene-1,14-diol, or other diols including macromonomer diols such as, for example polyethylene oxide, and N-methyldiethanoamine (MDEA) can also be used. Preferably, the polyol is substituted or unsubstituted glycerol.

Component B may be derived from a polyacid, such as a diacid or higher order acid A wide variety of diacid, or higher order acids, can be used. Exemplary acids include, but are not limited to, citric acid (3 carbons), glutaric acid (5 carbons), adipic acid (6 carbons), pimelic acid (7 carbons), sebacic acid (8 carbons), and azelaic acid (nine carbons). Exemplary long chain diacids include diacids having more than 10, more than 15, more than 20, and more than 25 carbon atoms. Non-aliphatic diacids can also be used. For example, versions of the above diacids having one or more double bonds can be used to produce polyol-diacid copolymers. Preferably the diacid is substituted or unsubstituted sebacic acid.

Several substituents, such as amines, aldehydes, hydrazides, acrylates and aromatic groups, can be incorporated into the carbon chain. Exemplary aromatic diacids include terephthalic acid and carboxyphenoxy-propane. The polyacids, e.g. the diacids, can also include substituents as well. For example, reactive groups like amine and hydroxyl can be used to increase the number of sites available for cross-linking. Amino acids and other biomolecules can be used to modify the biological properties. Aromatic groups, aliphatic groups, and halogen atoms can be used to modify the inter chain interactions within the polymer.

The pre-polymer may further comprise a polyamide or polyurethane backbone. For example, polyamine (comprising two or more amino groups) may be used to react with polyacid together with polyol or after reacting with polyol. In other examples, polyisocianates (comprising two or more isocyanate groups) may be used to react with polyacid together with polyol or after reacting with polyol.

The pre-polymer is preferably able to be activated. It can be activated by introducing functional groups that can react or be reacted to form crosslinks. Suitable functional groups to be activated on the pre-polymer backbone include hydroxy groups, carboxylic acid groups, amines, and combinations thereof, preferably hydroxy and/or carboxylic acid. The free hydroxyl or carboxylic acid groups on the pre-polymer can be activated by functionalizing the hydroxy groups with a moiety which can form a crosslink between polymer chains. The groups that are activated can be free hydroxyl or carboxylic acid groups on A and/or B moieties in the pre-polymer. Preferably, the functional group is or contains an acrylate group. Acrylate groups are moieties containing substituted or unsubstituted acryloyl group. The acrylate may contain the following group: —C(═O)—CR1=CR2R3, wherein R1, R2, R3 are independently from one another, selected in the group consisting of H, alkyl such as methyl or ethyl, aryl such as phenyl, substituted alkyl, substituted aryl, carboxylic acid, ester, amide, amine, urethane, ether, and carbonyl. Preferably, R1, R2 and R3 are H; or R1 is CH3, R2 and R3 are H; or R1 and R2 are H and R3 is CH3; or R1 and R2 are H and R3 is phenyl.

Preferably, at least a proportion of the activated groups (e.g., acrylate) on the polymeric backbone of the pre-polymer are reacted with a compound containing a charged atom, preferably a positively charged nitrogen atom.

Preferably, the medical adhesive is a light-curable compound. “Light curable compound” refers to compounds that are configured to polymerize or otherwise cure upon receiving appropriate radiant energy, more particularly in the form of light from a light source.

Preferably, the light-curable compound comprises a pre-polymer and a photoinitiator, said photoinitiator being able to induce polymerization of the said pre-polymer when exposed to light of a specific wavelength.

In some embodiments, said photoinitiator is sensitive to ultraviolet (UV) radiations. Examples of suitable photoinitiators sensitive to UV radiations include, but are not limited to: 2-dimethoxy-2-phenyl-acetophenone, 2-hydroxy-1-[4-(hydroxyethoxy)phenyl]-2-methyl-1-propanone (Irgacure 2959), 1-hydroxycyclohexyl-1-phenyl ketone (Irgacure 184), 2-hydroxy-2-methyl-1-phenyl-1-propanone (Darocur 1 173), 2-benzyl-2-(dimehylamino)-1-[4-morpholinyl)phenyl]-1-butanone (Irgacure 369), methylbenzoylformate (Darocur MBF), oxy-phenyl-acetic acid-2-[2-oxo-2-phenyl-acetoxy-ethoxy]-ethyl ester (Irgacure 754), 2-methyl-1-[4-(methylthio)phenyl]-2-(4-morpholinyl)-1-propanone (Irgacure 907), diphenyl(2,4,6-trimethylbenzoyl)-phosphine oxide (Darocur TPO), phosphine oxide, phenyl bis(2, 4, 6-trimethyl benzoyl) (Irgacure 819), and combinations thereof.

In some embodiments, said photoinitiator is sensitive to visible light (typically blue light or green light). Examples of photoinitiators sensitive to visible light include, but are not limited to: diphenyl(2,4,6-trimethylbenzoyl)-phosphine oxide, eosin Y disodium salt, N-Vinyl-2-Pyrrolidone (NVP) and triethanolamine, and camphorquinone.

In addition, the medical adhesive may also contain one or more pharmaceutical, therapeutic, prophylactic agents that can be released during the time period that the material functions as an adhesive. The agent may be a small molecule agent, for example having molecular weight less than 2000, 1500, 1000, 750, or 500 Da, a biomolecule, for example peptide, protein, enzyme, nucleic acid, polysaccharide, growth factors, cell adhesion sequences such as RGD sequences or integrins, extracellular matrix components, or combinations thereof. Exemplary classes of small molecule agents include, but are not limited to, anti-inflammatoires, analgesics, antimicrobial agents, and combinations thereof.

Preferably, the medical adhesive is or comprises poly glycerol sebacate acrylate (PGSA) or PGSAA (e.g., as described in WO2021078962).

### BRIEF DESCRIPTION OF THE DRAWINGS

The present disclosure will be more readily appreciated by reference to the following detailed description when being considered in connection with the accompanying drawings in which;

FIG. 1 shows a schematic depiction of a nerve conduit according to the invention in a longitudinal section;

FIG. 2 shows a schematic depiction of a nerve conduit according to the invention in a longitudinal section according to FIG. 1 with alternative end portions;

FIG. 3 shows a schematic depiction of the nerve conduit according to FIG. 2 in a side view from one end portion;

FIG. 4 schematically shows the nerve conduit according to FIG. 2 in a perspective view;

FIG. 5 shows a schematic depiction of a nerve conduit according to the invention in a longitudinal section according to another embodiment;

FIG. 6 shows a schematic depiction of a nerve conduit according to the invention in a longitudinal section according to another embodiment;

FIGS. 7 and 8 schematically depict a nerve conduit according to the invention having a star-shaped profile;

FIGS. 9 to 11 show schematic depictions of a nerve conduit according to the invention in a perspective side view and having end portions with alternative retention surfaces; and

FIG. 12 shows a schematic depiction of an undercut defined by the grooves according to FIG. 10.

### DETAILED DESCRIPTION OF PREFERRED EMBODIMENTS

In the following, the invention will be explained in more detail with reference to the accompanying figures. In the Figures, like elements are denoted by identical reference numerals and repeated description thereof may be omitted in order to avoid redundancies.

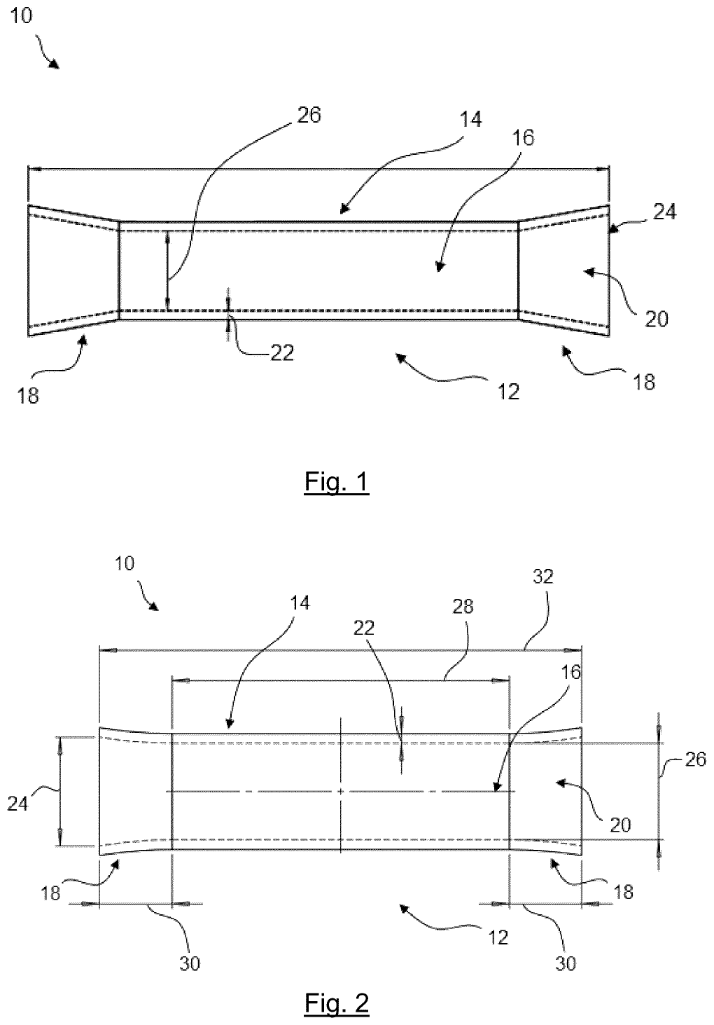

In FIG. 1 a nerve conduit 10 according to the invention is schematically shown along a longitudinal section. The nerve conduit 10 comprises an elongate body 12 having a central portion 14, which defines an inner cavity 16. In the present embodiment the inner cavity 16 is essentially defined by a wall 22. The wall 22 has a continuous tubular or cylindrical shape in the longitudinal direction of the elongate body 12 and has a continuous thickness both in the circumferential and longitudinal direction. Thereby, the inner cavity 16 is also formed as a cavity having a cylindrical shape having essentially continuous dimensions. However, it will be understood that embodiments of the invention are not limited to such configuration and varying wall thicknesses and/or alternative shapes off the central portion 14 and inner cavity 16 may also be provided.

The nerve conduit 10 or elongate body 12 thereof, according to the present exemplary embodiment, comprises two end portions 18, which are arranged at longitudinally opposing ends of the elongate body 12 and are arranged directly adjacent to the central portion 14. As indicated in the schematic depiction according to FIG. 1, the outer surfaces of both end portions 18 are aligned or flush with the outer surface of the central portion 14, such that a homogeneous and stepless outer surface is provided that is free of sharp edges, recesses, and/or protrusions potentially adversely affecting the surrounding tissue in the implanted state of the nerve conduit 10.

Both end portions 18 define an opening 20 to the inner cavity 16, such that a continuous through hole is provided from one end of the elongate body 12 to the opposing end of the elongate body 12 and a fluid communication between the inner cavity 16 and the exterior of the elongate body 12 is provided. Accordingly, nerve ends of a nerve to be repaired, e.g. after trauma resulting in a nerve lesion, may be inserted into the inner cavity 16 via a respective opening 20 of the corresponding end portion 18. The end portions 18 according to the present embodiment are equally shaped and dimensioned, such that during implantation a reversed orientation of the nerve conduit 10 does not affect the procedure.

The opening 20 of each of the end portions 18 increases along the longitudinal direction of the elongate body 12 in a direction away from the central portion 14. In other words, each opening 20 or radial extension thereof (gradually) increases starting from or extending from the central portion 14. In the present exemplary embodiment, a gradual increase of the openings 20 is provided by a conical shape of the end portions 18 or wall 22 thereof. The continuous wall thickness and the shapes of the central portion 14 and the end portions 18 hence provide that the cross-sectional area of each of the openings 20 is larger than the cross-sectional area of the inner cavity 16 of the central portion 14. Thereby, insertion of a respective nerve end into the inner cavity 16 and the application of a medical adhesive outside of the inner cavity 16, in particular at a junction or edge region of the opening, may be facilitated.

Both the inner cavity 16 and the openings 20 comprise an essentially circular shape in a cross-sectional view, wherein the diameter 26 of the inner cavity 16 is essentially continuous. The diameter of each of the openings 20 at the interface with the central portion 14 or inner cavity 16 essentially corresponds to the diameter 26 of the inner cavity 16. However, the diameter of each of the openings 20 increases along a longitudinal direction towards the outermost end of the elongate body 12 and end portion 18 respectively facing away from the central portion 14. In the present embodiment, the maximum diameter 24 and/or radial extension of each of the openings 20, and thereby the largest cross-sectional area, is hence provided at the longitudinally opposing and outermost ends of the elongate body 12. Having the largest cross-sectional area at the outermost end of the elongate body 16 further facilitates the insertion of a respective nerve end and/or the application of a medical adhesive outside of the inner cavity 16 or at the interface between the inner cavity 16 and the respective opening 20 or at the interface between the respective opening 20 and the respective inserted nerve end.

In FIG. 2, a nerve conduit 10 is shown, which essentially corresponds to the embodiment according to FIG. 1. However, this embodiment differs with regard to the shape of the end portions 18. In this embodiment, the end portions 18 are funnel-shaped and define a radially outward curvature or parabolic extension starting from the end facing the central portion 14 and extending towards the outermost end of the elongate body 12. In this manner, a larger opening 20 may be provided at the outermost end without requiring an increase in the length of the end portion 18 in the longitudinal direction and without requiring large angular offsets at the interface between the central portion 14 and the respective end portion 18, thereby also improving the structural stability of the nerve conduit 10. Furthermore, such shape may be advantageous to avoid sharp bends of the respective nerve end upon insertion and by providing a gradual guiding surface towards the inner cavity 16.

As indicated in FIG. 2 the maximum diameter 24 of the openings 20 is larger than the (constant) diameter 26 of the inner cavity 16. In addition, FIG. 2 demonstrates possible ratios between these diameters 24, 26 as well as length ratios between the corresponding portions 14, 18 of the elongate body 12.

For example, the ratio between the maximum diameter 24 of the opening 20 and the diameter 26 of the inner cavity 16 may be between 1.05:1.0 and 1.2:1.0. The maximum diameter 24 of the openings 20 may be from 1 mm to 15 mm, preferably from 1.5 mm to 13 mm, for example between 1.5 mm and 7.5 mm, more preferably from 1.75 mm to 7 mm, for example between 1.75 mm and 6.5 mm. The diameter 26 of the inner cavity 16 may be from 1 mm to 15 mm, preferably from 1 mm to 12 mm, for example between 1 mm and 12 mm or between 1.5 mm and 6.5 mm.

Accordingly, the cross-sectional area of the maximum diameter 26 of the openings may be from about 2.4 mm² to about 38.5 mm². The cross-sectional area of the diameter 26 of the inner cavity 16 may be from about 1.75 mm² to about 33.2 mm². In some embodiments, the maximum diameter 24 of the openings 20 may be between 1.75 mm and 6.5 mm or up to 12 mm while the diameter 26 of the inner cavity 16 may be between 1.5 mm and 6 mm or up to 11 mm. Accordingly, the cross-sectional area of the maximum diameter 26 of the openings may be between about 2.4 mm² and about 33.2 mm² and the cross-sectional area of the diameter 26 of the inner cavity 16 may be between about 1.75 mm² and about 28.3 mm².

In a particular example, the maximum diameter 24 may e.g. comprise 1.75 mm or 3.5 mm while the diameter 26 may accordingly comprise e.g. 1.5 mm and 3 mm, respectively, such that corresponding cross-sectional areas of the maximum diameter 24 may be about 2.4 mm² and about 9.6 mm² and corresponding cross-sectional areas of the diameter 26 of the inner cavity 16 may be about 1.75 mm² and about 7.1 mm² and a ratio of about 1.17:1.0 is obtained.

Such ratios have been found to be particularly advantageous to facilitate the insertion of respective nerve ends and/or apply a medical adhesive into the inner cavity 16 while limiting the radial extension of the nerve conduit. It is to be understood that embodiments may comprise the above configuration, but are not limited to the above exemplary dimensions and other dimensions or ratios may be provided as long as they are within the indicated preferred range.

Alternatively, or in addition, a ratio between the length 28 of the central portion 14 and the length 30 of an end portion 18 in the longitudinal direction may preferably be from 1.2:1.0 to 15:1.0; preferably from 1.2:1.0 to 12:1.0; more preferably from 1.2:1.0 to 10:1.0. In some embodiments, the ratio between the length 28 of the central portion 14 and the length 30 of an end portion 18 in the longitudinal direction is preferably between 1.2:1.0 and 1.4:1.0 or between 1.8:1.0 and 2.2:1.0 or between 4.4:1.0 and 5.4:1.0. By the same token, a ratio between the length 28 of the central portion 14 and the length 32 of the elongate body 12 in the longitudinal direction is preferably from 0.3:1.0 to 1:1.0; for example between 0.3:1.0 and 0.8:1.0. For example, the length 28 of the central portion 14 in the longitudinal direction may be from 3 mm to 40 mm, preferably from 4 mm to 30 mm, more preferably from 4.5 mm to 25 mm, for example between 6.5 mm and 8.5 mm. The length 32 of the elongate body in the longitudinal direction may be from 5 mm to 50 mm, preferably from 6 mm to 40 mm, more preferably from 7 mm to 30 mm, for example between 9 mm and 22 mm. The length 30 of the end portion 18 in the longitudinal direction may be from 1 mm to 8 mm, preferably between 1.3 mm and 6.5 mm.

In one exemplary embodiment, the length 28 of the central portion 14 may be e.g. 8 mm and the length 30 of the respective end portions 18 may be e.g. 6 mm, such that the total length 32 of the elongate body 12 may be e.g. 20 mm, resulting in a ratio of 1.33:1.0 and 0.4:1.0 between the length 28 of the central portion 14 and the length 30 of the respective end portion 18 and in a ratio of 0.4:1.0 between the length 28 of the central portion 14 and the length 32 of the elongate body 12. Again, it is to be understood that embodiments may comprise the above configuration, but are not limited to the above exemplary dimensions and other dimensions or ratios may be provided as long as they are within the indicated preferred range.

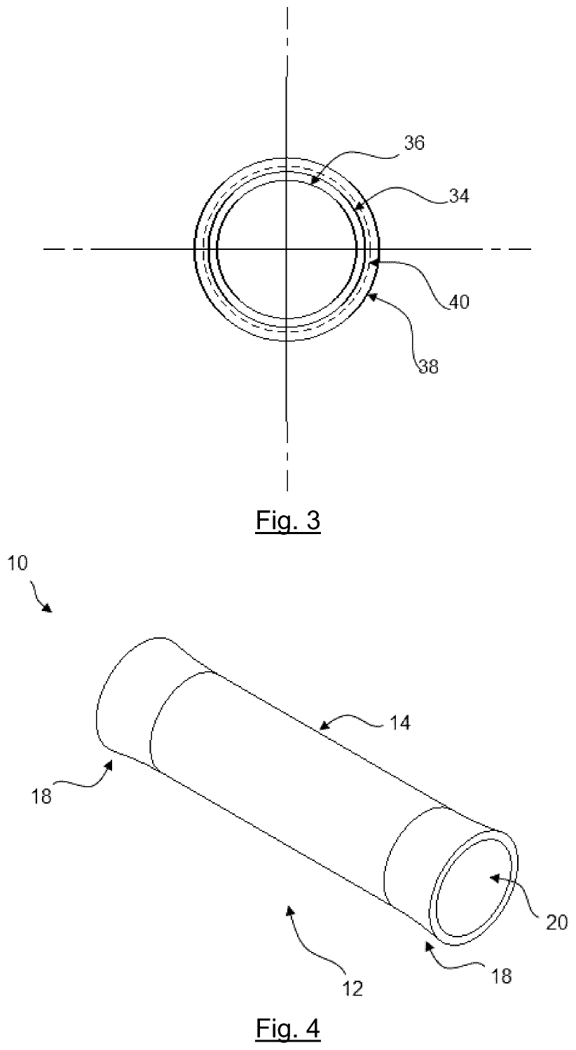

In FIG. 3 the different cross-sectional areas are shown in a side view of the nerve conduit as seen from one outermost end of the elongate body depicted in FIG. 2. Accordingly, the cross-sectional area 34 of the openings is larger than the cross-sectional area 36 of the inner cavity, which is indicated with the corresponding different diameters. In other words, the wall of the elongate body at the region defining the maximum diameter of the opening is radially offset to the inner wall of the central portion defining the inner cavity. As described above, the cross-sectional area of or at the maximum diameter of the openings may be e.g. between about 2.4 mm² and about 38.5 mm² and the cross-sectional area of the inner cavity 16 may be between about 1.75 mm² and about 33.2 mm². In some embodiments, the cross-sectional area of or at the maximum diameter of the openings may be e.g. between about 2.4 mm² and about 33.2 mm² and the cross-sectional area of the inner cavity 16 may be between about 1.75 mm² and about 28.3 mm². As indicated with the dashed line 40, the outer surface 38 of the end portion does not extend beyond the outer surface 40 of the central portion.

Although FIG. 3 comprises the cross-sectional area as a circular or round shape, it will be understood that the embodiments depicted in the Figures are not limited to such shape and other shapes such as ellipsoids may be provided, wherein the difference in the cross-sectional area 34 of the opening(s) and the cross-sectional area 36 of the inner cavity may be provided by an extension offset in at least one radial direction.

FIG. 4 schematically shows a nerve conduit 10 according to the embodiment shown in FIGS. 2 and 3 in a perspective view. From this Figure, it may be appreciated that the parabolic shape of the respective end portions 18 achieves that the opening 20 is increased towards the respective outermost ends of the elongate body 12 and provides a guiding surface facilitating insertion of the respective nerve ends into the inner cavity of the central portion 14. At the same time, the gradual increase of the opening 20 and the parabolic shape allow that the radial extension and the length of the end portions 18 may be kept to a minimum required and do not considerably affect the exterior and structural stability of the nerve conduit 10. Thereby, the repair of nerve lesions is facilitated by the advantageous configuration and dimensioning of the nerve conduit 10 according to the invention without adversely affecting the surrounding tissue in the implanted state of the nerve conduit 10.

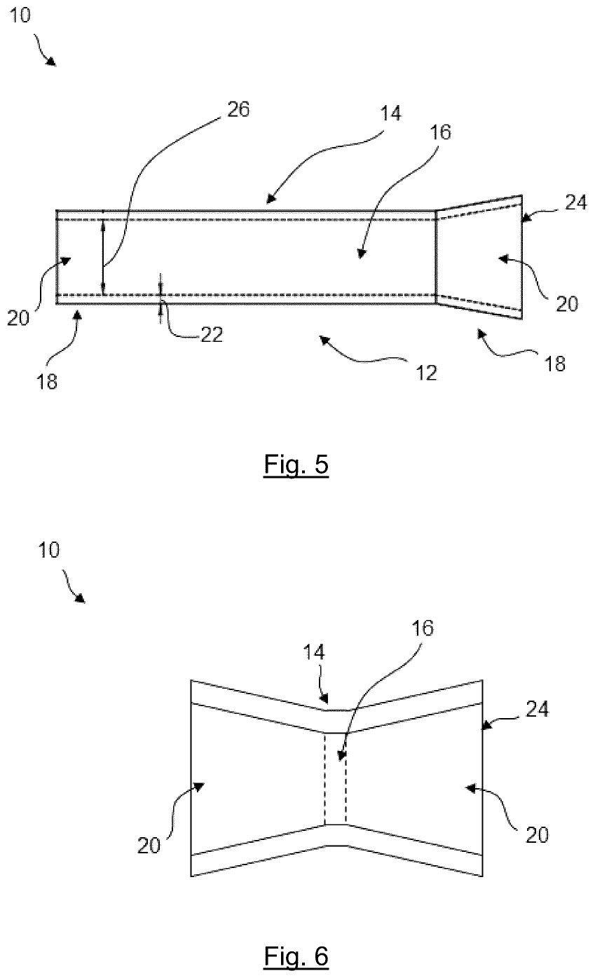

In FIG. 5, a nerve conduit 10 is schematically depicted according to an embodiment having distinct end portions 18 and openings 20, wherein only one opening 20 has a larger cross-sectional area compared with the central portion 14, i.e. having a larger maximum diameter 24 than the diameter 26 of the inner cavity 16. The nerve conduit 10 is hence formed as an asymmetric nerve conduit 10, which may particularly facilitate inserting a thicker nerve end at the opening 20 having the larger cross-sectional area.

An embodiment of a nerve conduit 10 having opposing end portions 18 and openings 20 that are formed of essentially the same shape, e.g. a funnel shape, is shown in FIG. 6, wherein the central portion 14 and inner cavity 16 are defined as an interface region between the respective openings 20, as indicated with the dashed lines. The interface region may e.g. be formed by an adjacent end of the respective openings 20 or end portions 18, which are adjoined and wherein the interface region may be rounded or formed so as to avoid significant steps between the respective end portions 18. In other words, the interface region may be formed to provide a continuous inner surface and outer surface of a wall essentially forming the nerve conduit 10. According to an embodiment, the longitudinal or axial extension of the central portion 14 is essentially negligible compared with the corresponding extension of the end portions 18 or openings 20, e.g. approximating zero or being less than e.g. 10 percent of the smallest axial extension of a respective opening 20 or end portion 18.

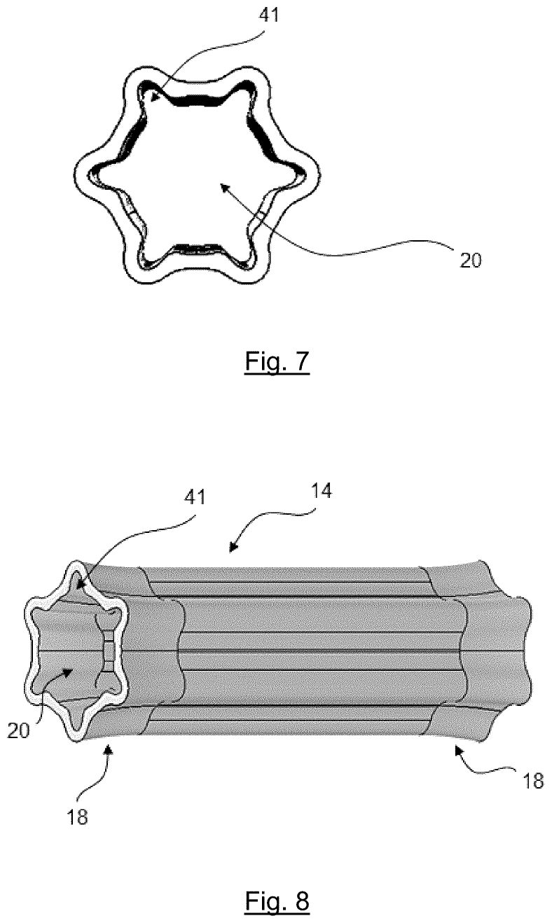

In FIGS. 7 and 8 a nerve conduit 10 according to the invention is shown having an elongate body with a star-shaped profile as seen in a cross-sectional view of the end portions 18 and which is provided in a rotationally symmetric manner. According to this exemplary embodiment, the wall of the elongate body comprises six rounded bulges 41 that radially extend the opening 20 and which are equally spaced apart to each other in the circumferential direction. The bulges 41 hence form inner and outer grooves, which are respectively defined by a circumferential width and radial extension of a respective bulge 41 or a spacing between two adjacent bulges 41. According to this example, each of the six bulges 41 extends along the circumference for about 20 degrees, such that a spacing between adjacent bulges 41 extends along the circumference for about 40 degrees.

As shown, the star shape extends throughout the entirety of the elongate body, including both the end portions 18 and the central portion 14. Furthermore, according to a preferred example, the star-shape of the elongate body is combined with a gradually increasing cross-sectional area of the openings 20, which comprise a funnel and/or parabolic shape at the respective end portion 18. The provision of such shape for the entirety of the elongate body facilitates handling during implantation and surgery while further improving the efficacy of a medical adhesive in securing a respective nerve end due to the provision of an increased contact surface at the respective end portion 18.

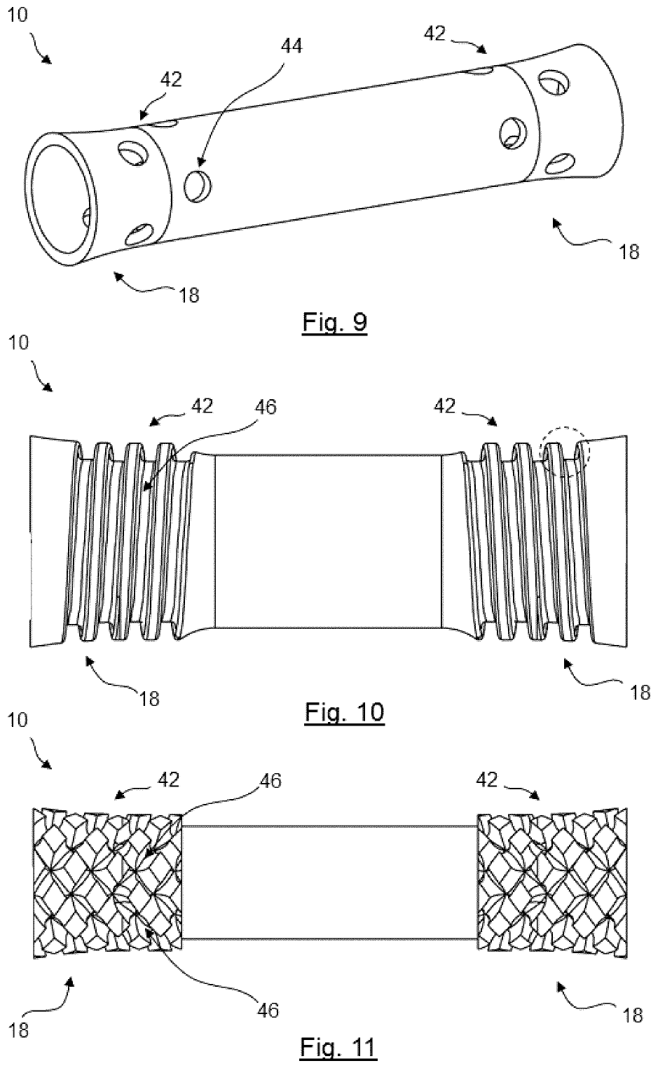

In FIGS. 9 to 11 a nerve conduit 10 according to the invention is shown schematically in a perspective side view, wherein the end portions 18 comprise alternative retention surfaces 42 configured to facilitate the securing of a medical adhesive to the respective end portion 18 at the outer surface of the elongate body 12. While some of these embodiments depict the retention surfaces 42 to be comprised, in part, on the central portion being directly adjacent to the respective end portion 18, it will be understood that such extension is merely optional.

Accordingly, FIG. 9 depicts a nerve conduit 10 having two opposing end portions 18, wherein both end portions 18 have a plurality of retention surfaces 42 in the form of holes 44. The holes 44 according to this preferred example are essentially circular yet being of slight ellipsoid shape, which may occur e.g. during manufacturing, and are formed as through-holes through a wall of the elongate body. In the exemplary embodiment, the holes 44 are arranged in two longitudinally spaced-apart rows in a circumferential direction of the elongate body, i.e. in a linear fashion along the circumference of the elongate body within each row and with a longitudinal offset of the rows to each other. As shown, one row of holes 44 is provided at the central portion being directly adjacent to the respective end portion 18 or at the interface between the central portion and the respective end portion 18. However, it will be understood that the holes 44 may also be exclusively at the respective end portion. Each row comprises six holes 44 that are equally spaced apart in the circumferential direction of the elongate body and are arranged in a staggered formation between the adjacent rows.

The embodiment according to FIG. 10 generally resembles the nerve conduit 10 according to FIG. 9. However, according to this embodiment, the retention surfaces 42 at the opposing end regions 18 are formed as grooves 46, which extend helically along an outer surface of the elongate body and along a longitudinal axis defined by said elongate body. The helical shape of the grooves 46 essentially defines a thread shape extending along the outer surface of the respective end portion 18. Said grooves 46 or threads are formed in a mirrored arrangement about a transverse mid plane of the elongate body, such that they are aligned in opposing directions, i.e. extending in clockwise and counter-clockwise direction. Although the number of revolutions may vary, the present embodiment depicts the grooves 46 having four revolutions, which has been found to be particularly advantageous in terms of facilitating the securing of the medical adhesive while maintaining structural stability and flexibility of the nerve conduit 10. The grooves 46 furthermore end at a longitudinal offset to an end surface of the respective end portion 18, which is furthermore advantageous in view of structural stability.

As will be shown in further detail below in view of FIG. 12, which is indicated by the dashed circle in FIG. 10, the grooves 46 furthermore comprise rounded edges and define an essentially continuous undercut, enhancing the efficacy of the medical adhesive by forming a form-fitting geometry.

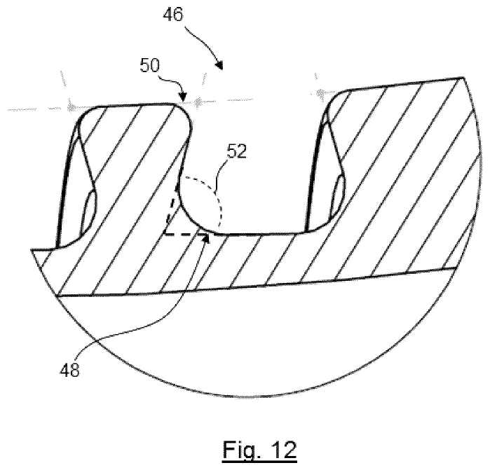

FIG. 11 depicts another embodiment of retention surfaces 42 at the opposing end portions 18, wherein each end portion 18 comprises a plurality of grooves 46 extending in opposing circumferential directions and intersecting each other, as indicated by the corresponding arrows. According to the embodiment, seven grooves 46 are provided in each circumferential direction at each respective end portion 18 and the number of grooves 46 is equal for each circumferential direction. As shown in FIG. 11, the plurality of grooves 46 in each circumferential direction together with their half-revolution along a longitudinal axis defined by the elongate body in a helical manner provides a diamond-shape or rhombic-shape at the outer surface of the respective end portion 18, which tends to resemble a pineapple surface. Thereby, a plurality of retention surfaces 42 with a large number of edges may be provided, which may be advantageous for securing the medical adhesive to the respective end portion 18.

FIG. 12 shows a schematic depiction of an undercut defined by the grooves 46 according to the dashed section indicated in FIG. 10 in a longitudinal section. As shown, a top outer surface of the groove 46 and a bottom surface of the groove 46, respectively corresponding to an outer radius 50 and an inner radius 48 and defining a groove depth, are rounded so as to reduce stress otherwise occurring in the material having grooves 46 with sharp edges. Furthermore, the top surface extends over the bottom surface in a longitudinal direction, thereby forming an angle 52 and corresponding undercut of the groove 46 at the bottom surface, as indicated by the dashed lines. The angle 52, preferably about 75 degrees, and corresponding undercut enable that a form-fitting or positive locking between an applied medical adhesive and the respective end portion 18 may be provided also in the radial direction, further improving the efficacy of the medical adhesive to secure a lesioned nerve end at the respective end portion 18.

It will be obvious for a person skilled in the art that these embodiments and items only depict examples of a plurality of possibilities. Hence, the embodiments shown here should not be understood to form a limitation of these features and configurations. Any possible combination and configuration of the described features can be chosen according to the scope of the invention.

The invention is furthermore depicted in the following items:
1. A nerve conduit (10), comprising an elongate body (12) comprising
   a central portion (14) defining an inner cavity (16) and
   end portions (18) defining a respective opening (20) to the inner cavity (16) and arranged adjacent to the central portion (14) and at longitudinally opposing ends of the elongate body (12),
   wherein a cross-sectional area of at least one opening (20) is larger than the cross-sectional area of the inner cavity (16) of the central portion (14).
2. The nerve conduit (10) according to item 1, wherein the central portion (14) is formed as an essentially tubular shape and the elongate body (12) comprises two end portions (18).
3. The nerve conduit according to item 1 or 2, wherein the openings (20) and inner cavity (16) define a single lumen or continuous through hole.
4. The nerve conduit (10) according to any of the preceding items, wherein the cross-sectional area of each of the openings (20) is larger than the cross-sectional area of the inner cavity (16) or wherein the cross-sectional area of an opening (20) of only one end portion (18) is larger than the cross-sectional area of the inner cavity (16).
5. The nerve conduit (10) according to any of the preceding items, wherein the cross-sectional area of the at least one opening (20) increases in the longitudinal direction and away from the central portion (14).
6. The nerve conduit (10) according to item 5, wherein the at least one end portion (18) and the corresponding opening (20) are formed as a rotationally symmetric shape along a longitudinal axis defined by the elongate body (12), said shape being an essentially U-shape, sigmoidal shape, conical shape, concave shape, funnel shape, or parabolic shape.
7. The nerve conduit (10) according to any of the preceding items, wherein the end portions (18) are equally formed.
8. The nerve conduit (10) according to any of the preceding items, wherein the inner cavity (16) and the openings (20) are formed by a single wall (22) of the elongate body (12) defining an inner diameter and outer diameter of the elongate body (12).
9. The nerve conduit (10) according to item 8, wherein the wall (22) comprises an essentially continuous thickness along the circumferential and longitudinal direction of the elongate body (12).
10. The nerve conduit (10) according to item 8 or 9, wherein the inner diameter and outer diameter of the central portion (14) are essentially continuous in the longitudinal direction of the elongate body (12).
11. The nerve conduit (10) according to any of items 8 to 10, wherein the diameter of the at least one opening (20) increases in the longitudinal direction away from the central portion (14).
12. The nerve conduit (10) according to item 11, wherein a ratio between the maximum diameter (24) of the at least one opening (20) and the diameter (26) of the inner cavity (16) is between 1.05:1.0 and 1.5:1.0, preferably between 1.05:1.0 and 1.2:1.0.
13. The nerve conduit (10) according to item 11 or 12, wherein the maximum diameter (24) of the at least one opening is smaller than or corresponds to the outer diameter of the central portion (14).
14. The nerve conduit (10) according to any of the preceding items, wherein the outer surface (38) of the end portions (18) extending from the central portion (14) is aligned with the outer surface (40) of the central portion (14) and/or is free of steps or edges with the outer surface (40) of the central portion (14).
15. The nerve conduit (10) according to any of the preceding items, wherein a ratio between the length (28) of the central portion (14) and the length (30) of an end portion (18) in the longitudinal direction is from 1.2:1.0 to 15:1.0; preferably from 1.2:1.0 to 12:1.0; more preferably from 1.2:1.0 to 10:1.0.
16. The nerve conduit (10) according to any of the preceding items, wherein a ratio between the length (28) of the central portion (14) and the length (30) of an end portion (18) in the longitudinal direction is between 1.2:1.0 and 6.0:1.0, preferably between 1.2:1.0 and 1.4:1.0 or between 1.8:1.0 and 2.2:1.0 or between 4.4:1.0 and 5.4:1.0.
17. The nerve conduit (10) according to any of the preceding items, wherein a ratio between the length (28) of the central portion (14) and the length (32) of the elongate body (12) in the longitudinal direction is from 0.3:1.0 to 1:1.0, for example between 0.3:1.0 and 0.8:1.0.
18. The nerve conduit (10) according to any of the preceding items, wherein the length (28) of the central portion (14) in the longitudinal direction is from 3 mm to 40 mm, preferably from 4 mm to 30 mm, more preferably from 4.5 mm to 25 mm.
19. The nerve conduit (10) according to any of the preceding items, wherein the length (32) of the elongate body (12) in the longitudinal direction is between 5 mm and 50 mm, preferably from 6 mm to 40 mm, more preferably from 7 mm to 30 mm.
20. The nerve conduit (10) according to any of the preceding items, wherein the length (30) of the end portion (18) in the longitudinal direction is from 1 mm to 8 mm, preferably between 1.3 mm and 6.5 mm.
21. The nerve conduit (10) according to any of the preceding items, wherein the length (28) of the central portion (14) in the longitudinal direction is between 5 mm and 10 mm, preferably between 6.5 mm and 8.5 mm, wherein the length (32) of the elongate body (12) in the longitudinal direction is between 7 mm and 25 mm, preferably between 9 mm and 22 mm, and/or wherein the length (30) of the end portion (18) in the longitudinal direction is between 1 mm and 8 mm, preferably between 1.3 mm and 6.5 mm.
22. The nerve conduit (10) according to any of the preceding items, wherein the diameter (26) of the inner cavity (16) is from 1 mm to 15 mm, preferably from 1 mm to 12 mm.
23. The nerve conduit (10) according to any of the preceding items, wherein the maximum diameter (24) of the at least one opening (20) is from 1 mm to 15 mm, preferably from 1.5 mm to 13 mm, more preferably from 1.75 mm to 7 mm.
24. The nerve conduit (10) according to any of the preceding items, wherein the diameter (26) of the inner cavity (16) is between 1 mm and 12 mm, preferably between 1.5 mm and 6.5 mm, and/or wherein the maximum diameter (24) of the at least one opening (20) is between 1.5 mm and 11 mm, preferably between 1.75 mm and 6.5 mm.
25. The nerve conduit (10) according to any of the preceding items, wherein the elongate body (12) is formed of a biocompatible material, an inert material, a bioimplantable material, and/or biodegradable material.
26. The nerve conduit (10) according to any of the preceding items, wherein the elongate body (12) is formed of a polymer-based material, preferably an elastomer.
27. The nerve conduit (10) according to any of the preceding items, wherein the central portion (14) and the end portions (18) are integrally formed or formed of a single piece.
28. The nerve conduit (10) according to any of the preceding items, wherein the elongate body (12) is formed of a polymerized and/or crosslinked polymer unit comprising an ester group component and an acid ester group component, the ester group component preferably being a polyol and the acid ester group component preferably being a polyacid.
29. The nerve conduit (10) according to any of the preceding items formed by a 3D-printing process.
30. Use of a nerve conduit (10) according to any of the preceding items for repairing, supporting, and/or guiding neural tissue, in particular for repairing a peripheral nerve lesion.
31. Use of a nerve conduit (10) according to item 30 in combination with a medical adhesive.
32. A method of treating a peripheral nerve lesion, comprising the steps of:
   providing a nerve conduit (10) according to any of the preceding claims;
   inserting one end of the lesioned nerve into the nerve conduit (10) via an opening (20) having a larger cross-sectional area than the inner cavity (16) of the central portion (14);
   inserting another end of the lesioned nerve via an opening (20) at an end portion (18) at a longitudinally opposing end of the elongate body (12); and
   securing the lesioned nerve ends within the elongate body (12).
27. The method according to item 32, wherein the securing of the lesioned nerve ends is performed by applying a medical adhesive outside of the inner cavity (16) and/or into the openings (20) via at least one of the openings (20) and/or around the openings (20).

### LIST OF REFERENCE NUMERALS

10 Nerve conduit
12 Elongate body
14 Central portion
16 Inner cavity
18 End portion
20 Opening
22 Wall
24 Maximum diameter of opening
26 Diameter of inner cavity
28 Length of central portion
30 Length of end portion
32 Length of elongate body
34 Cross-sectional area of opening
36 Cross-sectional area of inner cavity
38 Outer surface of end portion
40 Outer surface of central portion
41 Bulge
42 Retention surface
44 Hole
46 Groove
48 Inner radius
50 Outer radius
52 Angle

1. A nerve conduit, comprising:
an elongate body comprising;
a central portion defining an inner cavity; and
end portions each defining a respective opening to the inner cavity and arranged adjacent to the central portion and at longitudinally opposing ends of the elongate body;
wherein a cross-sectional area of at least one of the openings is larger than a cross-sectional area of the inner cavity of the central portion.

2. The nerve conduit according to claim 1, wherein the central portion is formed as an essentially tubular shape and the elongate body comprises two of the end portions.

3. The nerve conduit according to claim 1, wherein the openings and inner cavity define a single lumen or continuous through hole.

4. The nerve conduit according to claim 1, wherein the cross-sectional area of each of the openings is larger than the cross-sectional area of the inner cavity or wherein the cross-sectional area of an opening of only one end portion is larger than the cross-sectional area of the inner cavity.

5. The nerve conduit according to claim 1, wherein the cross-sectional area of the at least one opening increases in the longitudinal direction and away from the central portion.

6. The nerve conduit according to claim 5, wherein the at least one end portion and the corresponding opening are formed as a rotationally symmetric shape along a longitudinal axis defined by the elongate body, said shape being an essentially U-shape, sigmoidal shape, conical shape, concave shape, funnel shape, or parabolic shape.

7. The nerve conduit according to claim 1, wherein the end portions are equally formed.

8. The nerve conduit according to claim 1, wherein the inner cavity and the openings are formed by a single wall of the elongate body defining an inner diameter and outer diameter of the elongate body.

9. The nerve conduit according to claim 8, wherein the wall comprises an essentially continuous thickness along the circumferential and longitudinal direction of the elongate body.

10. The nerve conduit according to claim 8, wherein the inner diameter and outer diameter of the central portion are essentially continuous in the longitudinal direction of the elongate body.

11. The nerve conduit according to claim 8, wherein the diameter of the at least one opening increases in the longitudinal direction away from the central portion.

12. The nerve conduit according to claim 11, wherein a ratio between a maximum diameter of the at least one opening and the diameter of the inner cavity is preferably between 1.05:1.0 and 1.5:1.0, or between 1.05:1.0 and 1.2:1.0.

13. The nerve conduit according to claim 11, wherein a maximum diameter of the at least one opening is smaller than or corresponds to the outer diameter of the central portion.

14. The nerve conduit according to claim 1, wherein an outer surface of the end portions extending from the central portion is aligned with an outer surface of the central portion and/or is free of steps or edges with the outer surface of the central portion.

15. The nerve conduit according to claim 1, wherein a ratio between a length of the central portion and a length of one of the end portions in a longitudinal direction is from 1.2:1.0 to 15:1.0; or from 1.2:1.0 to 12:1.0; or from 1.2:1.0 to 10:1.0.

16. The nerve conduit according to claim 1, wherein a ratio between a length of the central portion and a length of one of the end portions in a longitudinal direction is between 1.2:1.0 and 6.0:1.0; or between 1.2:1.0 and 1.4:1.0; or between 1.8:1.0 and 2.2:1.0; or between 4.4:1.0 and 5.4:1.0.

17. The nerve conduit according to claim 1, wherein a ratio between a length of the central portion and a length of the elongate body in a longitudinal direction is from 0.3:1.0 to 1:1.0, or between 0.3:1.0 and 0.8:1.0.

18. The nerve conduit according to claim 1, wherein a length of the central portion in a longitudinal direction is from 3 mm to 40 mm, or from 4 mm to 30 mm, or from 4.5 mm to 25 mm.

19. The nerve conduit according to claim 1, wherein a length of the elongate body in a longitudinal direction is between 5 mm and 50 mm, or from 6 mm to 40 mm, or from 7 mm to 30 mm.

20. The nerve conduit according to claim 1, wherein a length of one of the end portions in a longitudinal direction is from 1 mm to 8 mm, or between 1.3 mm and 6.5 mm.

21. The nerve conduit according to claim 1, wherein a length of the central portion in a longitudinal direction is between 5 mm and 10 mm, or between 6.5 mm and 8.5 mm, wherein the length of the elongate body in the longitudinal direction is between 7 mm and 25 mm, or between 9 mm and 22 mm, and/or wherein the length of one of the end portions in the longitudinal direction is between 1 mm and 8 mm, or between 1.3 mm and 6.5 mm.

22. The nerve conduit according to claim 1, wherein the central portion is defined by an interface region between respective end portions and/or corresponding openings, wherein an extension of the central portion in a longitudinal direction is less than about 10 percent of a smallest extension of a respective opening and/or end portion in the longitudinal direction.

23. The nerve conduit according to claim 1, wherein a diameter of the inner cavity is from 1 mm to 15 mm, or from 1 mm to 12 mm.

24. The nerve conduit according to claim 1, wherein a maximum diameter of the at least one opening is from 1 mm to 15 mm, or from 1.5 mm to 13 mm, or from 1.75 mm to 7 mm.

25. The nerve conduit according to claim 1, wherein the diameter of the inner cavity is between 1 mm and 12 mm, preferably between 1.5 mm and 6.5 mm, and/or wherein the maximum diameter of the at least one opening is between 1.5 mm and 11 mm, preferably between 1.75 mm and 6.5 mm.

26. The nerve conduit according to claim 1, wherein the inner cavity of the central portion defines a single lumen and the nerve conduit comprises two of the end portions, which are located at either end of the central portion, wherein a maximum outer diameter of the end portions is larger than an outer diameter of the central portion and an outer end of the end portion is provided by a continuous edge.

27. The nerve conduit according to claim 1, wherein:
a cross-sectional area of the at least one opening increases in a longitudinal direction and away from the central portion;
the at least one end portion and the corresponding opening are formed as a rotationally symmetric funnel and/or parabolic shape along a longitudinal axis defined by the elongate body;
the inner cavity and the openings are formed by a single wall of the elongate body defining an inner diameter and outer diameter of the elongate body and having an essentially continuous thickness along the circumferential and longitudinal direction of the elongate body; and
the inner diameter and outer diameter of the central portion are essentially continuous in the longitudinal direction of the elongate body.

28. The nerve conduit according to claim 1, wherein;
a cross-sectional area of each of the openings is larger than a cross-sectional area of the inner cavity and increases in a longitudinal direction and away from the central portion;
the at least one end portion and the corresponding opening are formed as a rotationally symmetric funnel and/or parabolic shape along a longitudinal axis defined by the elongate body; and
the inner cavity and the openings are formed by a single wall of the elongate body having an essentially continuous thickness along the circumferential and longitudinal direction of the elongate body.

29. The nerve conduit according to claim 1, wherein at least one of the end portions comprises one or more retention surfaces at an outer surface of the elongate body and being configured for securing a medical adhesive to the respective end portion.

30. The nerve conduit according to claim 29, wherein the one or more retention surfaces of a respective end portion are formed as a plurality of ellipsoid or circular holes arranged in at least one row in a circumferential direction of the elongate body.

31. The nerve conduit according to claim 30, wherein the holes are arranged in 2 to 4 rows, wherein each row comprises 4 to 8 holes and wherein the holes of adjacent rows are arranged in a staggered formation.

32. The nerve conduit according to claim 30, wherein each hole is formed as a cut-out, a recess, or a through-hole in a wall forming the elongate body.

33. The nerve conduit according to claim 29, wherein the one or more retention surfaces of a respective end portion are formed as at least one groove extending in a helical direction along a longitudinal axis defined by the elongate body.

34. The nerve conduit according to claim 33, wherein the at least one groove comprises rounded edges and/or wherein the at least one groove defines at least one undercut.

35. The nerve conduit according to claim 33, wherein the at least one groove defines an outermost edge of the respective end portion in a longitudinal direction of the elongate body.

36. The nerve conduit according to claim 33, wherein the at least one groove comprises a radial depth varying in accordance with an increase of a cross-sectional area of the at least one opening in the longitudinal direction and away from the central portion.

37. The nerve conduit according to claim 33, wherein the at least one groove extends between 0.5 and 10 revolutions around a longitudinal axis defined by the elongate body.

38. The nerve conduit according to claim 37, wherein the at least one groove extends between 2 and 6 revolutions around the longitudinal axis defined by the elongate body.

39. The nerve conduit according to claim 37, wherein the respective end portion comprises at least two grooves extending in opposing circumferential directions and intersecting each other, each groove extending between 0.5 and 5 revolutions around a longitudinal axis.

40. The nerve conduit according to claim 29, wherein the one or more retention surfaces of a respective end portion are formed as one or more circumferential ribs extending from an outer surface of the elongate body.

41. The nerve conduit according to claim 40, wherein the extension of the one or more ribs is linear to a circumferential direction or comprises an offset to the circumferential direction in a longitudinal direction of the elongate body.

42. The nerve conduit according to claim 1, wherein the elongate body is formed of a biocompatible material, an inert material, a bioimplantable material, and/or biodegradable material.

43. The nerve conduit according to claim 1, wherein the elongate body is formed of a polymer-based material, or an elastomer.

44. The nerve conduit according to claim 1, wherein the central portion and the end portions are integrally formed or formed of a single piece.

45. The nerve conduit according to claim 1, wherein the elongate body is formed of a polymerized and/or crosslinked polymer unit comprising an ester group component and an acid ester group component, the ester group component being a polyol and the acid ester group component being a polyacid.

46. The nerve conduit according to claim 1 formed by a 3D-printing process.

47. Use of a nerve conduit according to claim 1 for repairing, supporting, and/or guiding neural tissue, or for repairing a peripheral nerve lesion.

48. (canceled)

49. A method of treating a peripheral nerve lesion, comprising the steps of:
providing a nerve conduit according to claim 1;
inserting one end of the lesioned nerve into the nerve conduit via an opening having a larger cross-sectional area than the inner cavity of the central portion;
inserting another end of the lesioned nerve via an opening at an end portion at a longitudinally opposing end of the elongate body; and
securing the lesioned nerve ends within the elongate body.

50. The method according to claim 49, wherein the securing of the lesioned nerve ends is performed by applying a medical adhesive outside of the inner cavity and/or into the openings via at least one of the openings and/or around the openings.
